(19) United States
(12) Patent Application Publication
(10) Pub. No.: US 2016/0290936 A1
(43) Pub. Date: Oct. 6, 2016

(54) TRANSPARENT TYPE FLAT PANEL X-RAY GENERATION APPARATUS AND X-RAY IMAGING SYSTEM

(71) Applicant: SAMSUNG ELECTRONICS CO., LTD., Suwon-si, Gyeonggi-do (KR)

(72) Inventors: Taewon JEONG, Yongin-si (KR); Doyoon KIM, Hwaseong-si (KR); Ilhwan KIM, Yongin-si (KR); Shanghyeun PARK, Yongin-si (KR); Changsoo LEE, Seoul (KR); Yongchul KIM, Seoul (KR)

(73) Assignee: Samsung Electronics Co., Ltd., Suwon-si, Gyeonggi-do (KR)

(21) Appl. No.: 15/034,337

(22) PCT Filed: Nov. 3, 2014
(86) PCT No.: PCT/KR2014/010444

§ 371 Date: May 4, 2016

(30) Foreign Application Priority Data
Nov. 5, 2013 (KR) 10-2013-0133826

(51) Int. Cl. G01N 23/04; H01J 35/18; H01J 35/06; H01J 35/08

(52) U.S. Cl. CPC G01N 23/04; H01J 35/08; H01J 35/18; H01J 35/06; H01J 2235/088; H01J 2235/1204

(57) ABSTRACT

An X-ray generation apparatus includes: an electron emission device comprising a plurality of electron emission units that emit electrons; a transmission type X-ray emission unit for emitting an X-ray by electrons emitted by the plurality of electron emission units; and a vacuum chamber for shielding the electron emission device and the transmission type X-ray emission unit by using vacuum. An X-ray imaging system includes an X-ray detection apparatus for detecting an X-ray that is irradiated from the X-ray generation apparatus and passes through an object.

### TECHNICAL FIELD

The present disclosure relates to a transparent type flat panel X-ray generation apparatus and an X-ray imaging system.

### BACKGROUND ART

X-rays are used in non-destructive testing, structural and physical properties testing, image diagnosis, security inspection, and the like in the fields of industry, science, medical treatment, etc. Generally, an imaging system using X-rays for such purposes includes an X-ray generation apparatus for radiating an X-ray and an X-ray detection apparatus for detecting an X-ray that have passed through an object.

The X-ray detection apparatus is being rapidly converted from a filming method to a digitalization method, whereas the X-ray generation apparatus uses an electron generation device using a tungsten filament type cathode. Thus, a single electron generation device is mounted in a single X-ray photographing device.

### DISCLOSURE OF INVENTION

### Technical Problem

Meanwhile, the X-ray detection apparatus is generally implemented in a flat panel type, which problematically causes a predetermined distance between the X-ray generation apparatus and the object so as to obtain an image from the single electron generation device.

Furthermore, the object having a predetermined area needs to be photographed from a single X-ray generation apparatus, which makes it impossible to select and photograph a specific part of the object.

The X-ray generation apparatus has problems of generating a great amount of heat when dissipating X-ray and having a low X-ray transmittance.

### Solution to Problem

Provided are a transparent type flat panel X-ray generation apparatus and an X-ray imaging system.

Additional aspects will be set forth in part in the description which follows and, in part, will be apparent from the description, or may be learned by practice of the presented embodiments.

According to an aspect of the present invention, an X-ray generation apparatus includes: an electron emission device including a plurality of electron emission units that are independently driven and emit electrons; a transmission type X-ray emission unit for emitting an X-ray by electrons emitted by the plurality of electron emission units; and a vacuum chamber for shielding the electron emission device and the transmission type X-ray emission unit by using vacuum.

An X-ray transmission window that radiates the X-ray emitted by the X-ray emission unit to the outside of the vacuum chamber may be provided in the vacuum chamber. The X-ray transmission window may include Be, C, Al, or a metal alloy including at least one of Be, C, and Al.

One or more of the plurality of electron emission units may be simultaneously or sequentially driven to emit the electrons. The X-ray emission unit may include a plurality of X-ray emitters that emit the X-ray by the electrons emitted by the plurality of electron emission units.

The X-ray emission unit may include an anode electrode that generates the X-ray by the electrons emitted by the plurality of electron emission units, wherein the anode electrode comprises an anode substrate and a coating layer provided on one surface of the anode substrate. The coating layer may include W, Mo. Ag, Cr, Fe, Co, Cu or a metal alloy including at least one of W, Mo. Ag, Cr, Fe, Co, and Cu.

The anode substrate may be a carbon substrate. The coating layer may be a tungsten coating layer.

The X-ray generation apparatus may further include: an insulating layer for separating the X-ray emission unit from the vacuum chamber.

The X-ray generation apparatus may further include: a cooling apparatus spaced apart from the vacuum chamber by an insulating layer and cooling heat generated by the anode electrode.

The insulating layer may include ceramics or plastics.

The vacuum chamber may include a getter pump that removes a gas from the inside thereof.

Each of the plurality of electron emission units may include: a gate insulating layer provided on a substrate and forming a cavity connected to the outside by an opening through which the electrons are emitted; a cathode electrode disposed in the cavity; an electron emission source disposed on the cathode electrode; and a gate electrode on the gate insulating layer.

According to another aspect of the present invention, an X-ray imaging system includes: the above-described X-ray generation apparatus; and the above mentioned X-ray detection apparatus for detecting an X-ray that is irradiated from the X-ray generation apparatus and passes through an object.

The X-ray detection apparatus may include a plurality of X-ray detection units that are 2-dimensionally arranged and independently driven.

### Advantageous Effects of Invention

The X-ray imaging system according to the above-described embodiment includes a flat panel type X-ray generation apparatus. Thus, an object is disposed between the flat panel type X-ray generation apparatus and an X-ray detection apparatus, thereby implementing the X-ray imaging system having a very small thickness. A limited part of the X-ray generation apparatus may be partially driven to generate an X-ray, thereby photographing a specific region of the object, and such selectively partial photographing prevents an irradiation of the X-ray to an unnecessary region, thereby reducing an exposure rate. The X-ray generation apparatus may include an electron emission device including a plurality of electron emission units that are independently driven. The X-ray generation apparatus may include a plurality of X-ray emission units that are independently driven and generate the X-ray by electrons emitted by the electron emission device. At least a part of the plurality of electron emission units may be sequentially driven, thereby 3-dimensionally photographing the specific region of the object. A cooling apparatus may be used to reduce heating in an anode electrode to which a high voltage is applied, and a high voltage stability may increase in proximity photographing of the object by using an insulating layer. A transmission loss may be reduced through an X-ray transmission window provided in a vacuum chamber.

### BRIEF DESCRIPTION OF DRAWINGS

These and/or other aspects will become apparent and more readily appreciated from the following description of the embodiments, taken in conjunction with the accompanying drawings in which:

FIG. 1 is a cross-sectional view of an X-ray generation apparatus, according an exemplary embodiment;

FIG. 2 is a cross-sectional view of an X-ray generation apparatus, according another exemplary embodiment;

FIG. 3 is a schematic cross-sectional view of an electron emission device of FIGS. 1 and 2;

FIG. 4 is a schematic plan view of an example of a driving wire of the electron emission device of FIGS. 1 and 2; and

FIG. 5 is a cross-sectional view of an X-ray imaging system according an exemplary embodiment.

### MODE FOR THE INVENTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as at least one of, when preceding a list of elements, modify the entire list of elements and do not modify the individual elements of the list.

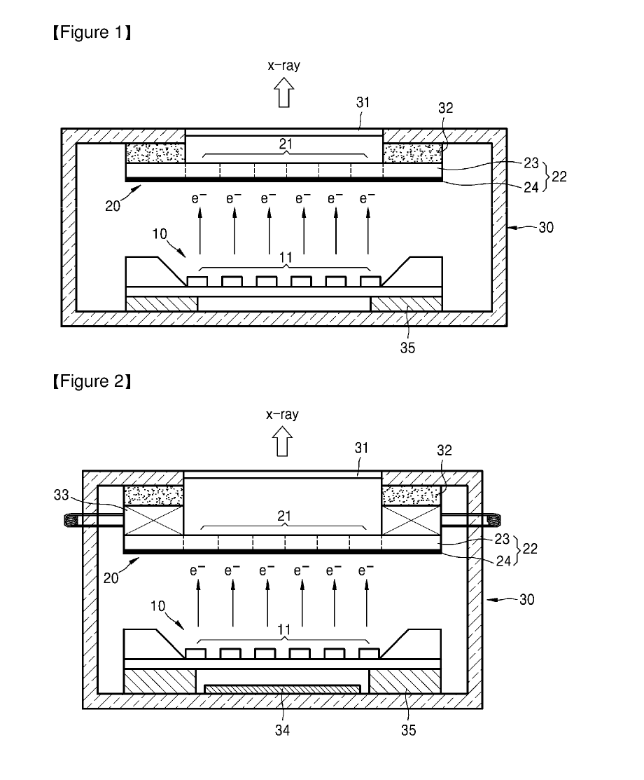

FIG. 1 is a cross-sectional view of an X-ray generation apparatus 100, according an exemplary embodiment. FIG. 2 is a cross-sectional view of the X-ray generation apparatus 100, according another exemplary embodiment.

Referring to FIGS. 1 and 2, the X-ray generation apparatus 100 includes an electron emission device 10 including a plurality of electron emission units 11 that may be independently driven and emit electrons e⁻, an X-ray emission unit 20 that emits an X-ray by the electrons e^{—} emitted by the electron emission units 11, and a vacuum chamber 30 that shields the electron emission device 10 and the an X-ray emission unit 20 in a vacuum way.

The electron emission units 11 may be wholly or partly driven. Thus, an irradiation range of the X-ray to an object may be adjusted by simultaneously or sequentially driving one or more of the electron emission units 11 (i.e., selectively driving the electron emission units 11 wholly or partly). The electron emission units 11 may be classified into electron emission modules each including the one or more electron emission units 11. The X-ray may be irradiated to some regions of the object or may be sequentially irradiated to a plurality of regions of the object by simultaneously or sequentially driving the electron emission modules.

The X-ray emission unit 20 is a transparent type flat panel X-ray emission unit 20. The X-ray emission unit 20 is divided into a plurality of X-ray emitters 21. If the X-ray emitters 21 are wholly or partly driven, the electrons e⁻ emitted by the driven electron emission units 11 arrive at the X-ray emitters 21 corresponding to the driven electron emission units 11. The X-ray is emitted from the X-ray emitters 21 corresponding to the driven electron emission units 11.

Therefore, the irradiation range of the X-ray to the object may be adjusted by the electron emission units 11. For example, the irradiation range of the X-ray to the object may be adjusted by selectively driving the electron emission units 11 wholly or partly. The X-ray emitters 21 and the electron emission units 11 may correspond to each other one-to-one or, each of the X-ray emitters 21 may correspond to two or more of the electron emission units 11. Each of two or more of the X-ray emitters 21 may also correspond to each of the electron emission units 11.

The flat panel type X-ray generation apparatus 100 of the above-described structure may perform proximity photographing on the object, thereby minimizing a system size, and may generate a selective partial X-ray, thereby reducing an X-ray exposure of an unnecessary part. The flat panel type X-ray generation apparatus 100 may increase a uniformity of the X-ray higher than 90% by employing the transparent type flat panel X-ray emission unit 20.

The vacuum chamber 30 may be made of a metallic material that may endure an atmospheric pressure, for example, stainless steel, an aluminum alloy, etc.

The vacuum chamber 30 includes an X-ray transmission window 31 that radiates the X-ray emitted by the X-ray emission unit 20 to the outside of the vacuum chamber 30. To increase transmission efficiency, the X-ray transmission window 31 may use a material having a low atom number such as beryllium (Be) and these alloys. For example, the X-ray transmission window 31 includes Be, C, Al, or a metal alloy including at least one of these metals.

An X-ray transmittance loss may be reduced by using the X-ray transmission window 31 including the above-described material. An intensity Ix of the X-ray that transmits the X-ray transmission window 31 may be determined according to an equation below,

Iₓ=I₀ exp(−μρd)

wherein, I₀ denotes an initial intensity of the X-ray, μ denotes an X-ray absorption coefficient (cm²/g) of a material, ρ denotes a density (g/cm³) of a material through which the X-ray transmits, and d denotes a thickness (cm) of a region through which the X-ray transmits.

The X-ray absorption coefficient μ(cm²/g), the density ρ(g/cm³), and a melting point of the materials are shown in a table 1 below.

TABLE 1

| | μ (cm²/g) | ρ (g/cm³) | melting point (° C.) |
|---|---|---|---|
| Be | 0.014 | 1.85 | 1287 |
| C (graphite) | 0.024 | 2.09 | 3652 |
| Al | 0.184 | 2.70 | 660 |

As shown in the table 1 above, beryllium (Be) has a low X-ray absorption rate compared to carbon or aluminum, and thus the X-ray generation apparatus 100 having a very low transmission loss of the X-ray transmission window 31 may be implemented if beryllium (Be) is used.

The X-ray emission unit 20 includes an anode electrode 22 that generates the X-ray by the electrons e⁻ emitted by the electron emission device 10. The anode electrode 22 may include a coating layer 24 that generates the X-ray by the electrons e⁻. The coating layer 24 may include, for example, a metal such as W, Mo, Ag, Cr, Fe, Co, or Cu and a metal alloy including at least one of these metals. The anode electrode 22 may further include an anode substrate 23 that supports the coating layer 24. The anode substrate 23 may be formed of a material having transmittance with respect to the X-ray. The anode substrate 23 may be, for example, a glass substrate, a carbon substrate, etc. The coating layer 24 may be provided on a surface of the anode substrate 23 facing the electron emission device 10. A thickness of the coating layer 24 may be, for example, equal to or smaller than 5 um.

The anode electrode 22 may be a single flat panel or may be divided into a plurality of anode electrodes to correspond to the X-ray emitters 21.

According to an embodiment, the anode electrode 22 may include the carbon anode substrate 23 and the tungsten coating layer 24 provided on one surface of the carbon anode substrate 23. Tungsten (W) has a high melting point (3422 C) and an excellent X-ray generation characteristic. Carbon (graphite) has a relatively high melting point (about 3600 C) and a good X-ray transmittance. Thus, high X-ray generation efficiency and X-ray transmittance may be obtained, and a high thermal stability may be achieved by employing the carbon anode substrate 23 having the tungsten coating layer 24 as the anode electrode 22.

The X-ray emission unit 20 may be separated from the vacuum chamber 30 by using an insulating layer 32. The anode electrode 22 may be spaced apart from the vacuum chamber 30 by using the insulating layer 32. A high anode voltage is applied to the anode electrode 22 to pull at the electrons e⁻ emitted by the electron emission device 10. The anode electrode 22 is electrically insulated from the vacuum chamber 30 by using the insulating layer 32, thereby preventing a short circuit through the vacuum chamber 30. A high voltage stability may be secured in proximity photographing of the object. The insulating layer 32 may be, for example, ceramics such as alumina (Al₂O₃) or electrically insulating plastics.

The anode electrode 22 may generate a great amount of heat due to heating caused by collisions of the electrons e⁻ emitted by the electron emission device 10, resistive heating caused by a high anode voltage, etc. Such heat may deteriorate the X-ray generation efficiency, and badly influences a structural stability of the X-ray generation apparatus 100. To solve these problems, as shown in FIG. 2, a cooling apparatus 33 for cooling the anode electrode 22 is provided. The cooling apparatus 33 discharges the heat of the anode electrode 22 to the outside of the vacuum chamber 33. The cooling apparatus 33 may be separated from the vacuum chamber 30 by using the insulating layer 32 when the insulating layer 32 is provided. The cooling apparatus 33 may be an air-cooled, water-cooled, or electrical cooling apparatus. For example, a heat pipe may be employed as the cooling apparatus 33. The heat pipe has a structure in which a liquid (working fluid) is sealed in a vacuum pipe. One end (a heating portion) of the vacuum pipe absorbs heat from a cooled object. The liquid is evaporated by the heat. A vapor moves to another end (a condensation portion) of the vacuum pipe by a vapor pressure. At the other end of the vacuum pipe, the vapor condenses into the liquid again through a heat exchange with the outside and the condensed liquid return to one end by a capillary pressure along a wick provided inside of the vacuum pipe. Such a natural circulation of the working fluid may produce a cooling effect.

When the X-ray generation apparatus 100 operates, a gas may be generated in the vacuum chamber 30. The inside of the vacuum chamber 30 may be necessarily maintained in a high vacuum state. To this end, a vacuum pump 34 is disposed in the vacuum chamber 30. For example, a getter pump that absorbs the gas may be employed as the vacuum pump 34. The gas is mainly generated around the electron emission device 10 of the X-ray generation apparatus 100, and thus the getter pump may be disposed near the electron emission device 10. However, the scope of the present invention is not limited thereto. The getter pump may be formed in an inner wall of the vacuum chamber 30 excluding the X-ray transmission window 31. The getter pump may be implemented by heating and evaporating a getter material, such as barium, magnesium, zirconium, or an alloy of these, at a vacuum state and forming a getter material deposition film on the inner wall of the vacuum chamber 30.

Meanwhile, although not shown in FIGS. 1 and 2, a collimator for adjusting a direction of the X-ray may be provided between the X-ray generation apparatus 100 and the X-ray detection apparatus 200.

The electron emission device 10 is spaced apart from an inner wall surface of the vacuum chamber 30. For example, a support portion 35 may be disposed between the electron emission device 10 and the vacuum chamber 30. The support portion 35 may be, for example, an electrical insulator.

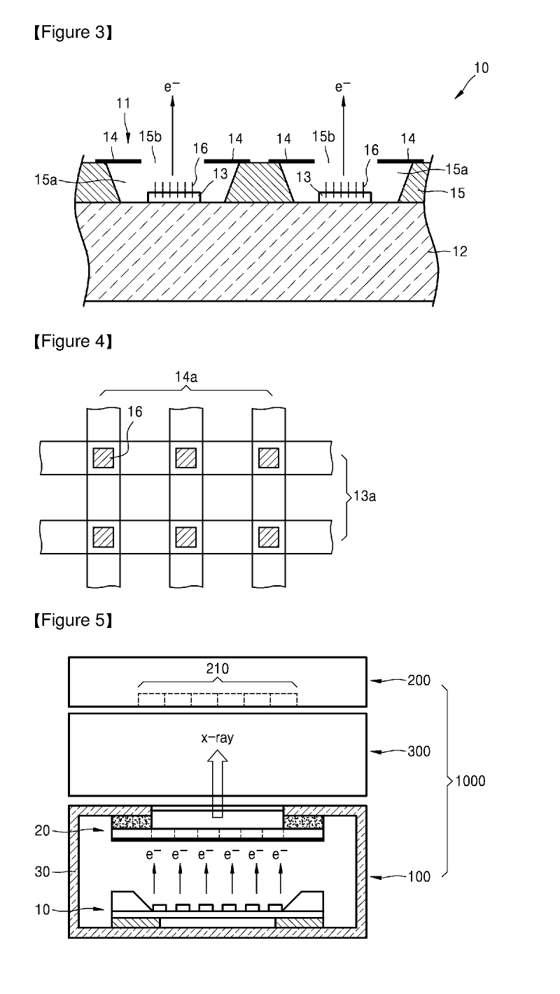

FIG. 3 is a schematic cross-sectional view of the electron emission device 10 of FIGS. 1 and 2. FIG. 4 is a schematic plan view of an example of a driving wire of the electron emission device 10 of FIGS. 1 and 2.

Referring to FIGS. 3 and 4, the electron emission device 10 has a structure in which the electron emission units 11 are 2-dimensionally aligned. Each of the electron emission units 11 includes a cathode electrode 13 and an electron emission source 16 that form an emitter that emits electrons, and a gate electrode 14. One emitter and one gate electrode 14 are disposed in each of the electron emission units 11 in FIGS. 3 and 4, but are not limited thereto. Two or more emitters and two or more gate electrodes 14 may be disposed in each of the electron emission units 11.

A plurality of cathode electrodes 13 to which voltages are applied through a plurality of cathode lines 13a of FIG. 4 are aligned on a substrate 12. The gate electrodes 14 to which voltages are applied through a plurality of gate lines 14a of FIG. 4 are aligned above the cathode electrodes 13 to correspond to the cathode electrodes 13. The cathode lines 13a and the gate lines 14a may be aligned to cross each other. The electron emission sources 16 are disposed at position in which the cathode lines 13a and the gate lines 14a cross each other so that the electron emission sources 16 may be aligned in a 2D matrix on the substrate 12. That is, the electron emission sources 16 may be aligned in a matrix of m×n (m and n are natural numbers equal to or greater than 2). The 2D aligned electron emission sources 16 may be independently driven to emit the electrons e⁻. That is, if a predetermined voltage is applied to each of one of the cathode lines 13a and one of the gate lines 14a, the electron emission source 16 that is disposed at a position in which the cathode line 14a and the gate line 14a to which the predetermined voltages are applied cross each other may be driven and emit the electrons e⁻.

Referring to FIG. 3, the cathode electrodes 13 are provided on the substrate 12. In this regard, an insulating substrate such as a glass substrate may be used as the substrate 12. However, the present invention is not necessarily limited thereto, and a conductive substrate may be used as the substrate 12. In this case, an insulating layer (not shown) may be formed on a surface of the conductive substrate. The cathode electrodes 13 may include a conductive material. For example, the cathode electrodes 13 may include metal or a conductive metal oxide. More specifically, the cathode electrodes 13 may include metal such as Ti, Pt, Ru, Au, Ag, Mo, Al, W, or Cu or a metal oxide such as indium tin oxide (ITO), aluminum zinc oxide (AZO), indium zinc oxide (IZO), SnO₂, or In₂O₃. However, this is merely an example, and the cathode electrodes 13 may include other diverse materials.

A gate insulating layer 15 is provided on the substrate 12. The gate electrodes 14 are provided on the gate insulating layer 15. The gate insulating layer 15 functions as insulating the cathode electrodes 13 and the gate electrodes 14 and simultaneously supporting the gate electrodes 14. The gate insulating layer 15 may include, for example, SiO₂, Si₃N₄, HfO₂, or Al₂O₃ but is not limited thereto. The gate electrodes 14 may be metal mesh electrodes. The gate electrodes 14 may include a conductive material like the cathode electrodes 13. For example, the gate electrodes 14 may include metal or a conductive metal oxide.

According to the above-described structure, an X-ray generation apparatus including the electron emission units 11 which are independently driven may be implemented.

A plurality of cavities 15a are formed on the substrate 12 by using the gate insulating layer 15. The cavities 15a are connected to the outside through openings 15b. The gate electrodes 14 are disposed to surround the openings 15b. The openings 15b are emission paths of the electrons e⁻. The cathode electrodes 13 and the electron emission sources 16 are positioned in the cavities 15a. The electron emission sources 16 may be provided on the cathode electrodes 13 in the cavities 15a. The electron emission sources 16 may be formed to be lower than the gate insulating layer 13. A voltage is applied between the cathode electrodes 13 and the gate electrodes 14 so that a strong electric field is applied to the electron emission sources 16, and the electrons e⁻ are emitted from the electron emission sources 16 by an energy provided by the electric field. The electrons e⁻ are moved to the anode electrode 22 of FIGS. 1 and 2 through the openings 15b. The electron emission sources 16 may include, for example, a carbon nanotube (CNT), a carbon nanofiber, metal, silicon, an oxide, diamond, diamond like carbon (DLC), a carbide compound or a nitrogen compound. However, the present invention is not limited thereto. Density of the electrons e⁻ emitted by the electron emission sources 16 is proportional to intensity of voltages applied to the gate electrodes 14. The higher the aspect ratio of the electron emission sources 16, the more the electric field enhancement effect that an electric field is focused in the electron emission sources 16 is obtained, and thus the density of the electrons e⁻ increases. Thus, the electron emission sources 16 may be disposed on the cathode electrodes 13, for example, in a sharp needle shape or a standing thin plate shape.

The cavities 15a may be formed to be wider toward upper portions thereof. The cavities 15a may have cross-sections of diverse shapes. An example case of the cavities 15a having rectangular cross-sections is shown in FIGS. 3 and 4. The cavities 15a may have circular cross-sections or cross-sections of other diverse shapes.

Referring to FIG. 4, the electron emission units 11 have a 2D matrix (X>Y matrix) in which horizontal lines are the cathode lines 13a and vertical lines are the gate lines 14a. That is, the cathode lines 13a that are the horizontal lines correspond to the cathode electrodes 13 of FIG. 3 and the gate lines 14a that are the vertical lines correspond to the gate electrodes 14 of FIG. 3. The cathode lines 13a and the gate lines 14a are separated from each other by an insulating layer. The insulating layer corresponds to the gate insulating layer 15 of FIG. 3.

To drive a specific cell, for example, a 3×2 cell (i.e. a cell positioned at a 3^{rd} in a lateral direction and a 2^{nd} in a transversal direction), voltages are applied to a 3^{rd} gate line and a 2^{nd} cathode line, an electric potential is induced in the 3×2 cell, and electrons are emitted from the electron emission source 16 corresponding to the 3×2 cell.

FIG. 5 is a cross-sectional view of an X-ray imaging system 1000 according an exemplary embodiment.

Referring to FIG. 5, the X-ray imaging system 1000 includes the flat panel type X-ray generation apparatus 100 and the X-ray detection apparatus 200 that detects an X-ray generated by the X-ray generation apparatus 100. Although the X-ray imaging system 1000 of FIG. 5 employs the X-ray generation apparatus 100 of FIG. 1 as the X-ray generation apparatus 100, the X-ray generation apparatus 100 of FIG. 2 may be employed.

An object 300 is disposed between the X-ray generation apparatus 100 and the X-ray detection apparatus 200. The X-ray detection apparatus 200 detects an X-ray that is emitted from the X-ray generation apparatus 100 and transmits the object 300 so that the inside of the object 300 may be photographed. The object 300 may be provided to contact the X-ray generation apparatus 100 and the X-ray detection apparatus 200. Meanwhile, the object 300 may be provided to contact the X-ray generation apparatus 100 or the X-ray detection apparatus 200.

The X-ray generation apparatus 100 may include the electron emission device 10 including the plurality of electron emission units 11 that are 2-dimensionally arranged and independently emit electrons. The X-ray generation apparatus 100 may include the X-ray emission unit 20 including the plurality of X-ray emitters 21 that are independently driven. The X-ray emission unit 20 emits the X-ray by the electrons emitted by the electron emission device 10. The X-ray emission unit 20 may be the transmission type X-ray emission unit 20 that receives the electrons, transmits the X-ray, and emits the X-ray to the outside. The electron emission device 10 includes the plurality of electron emission sources 16 that emit the electrons when an electric field is applied. The X-ray emission unit 20 includes the anode electrode 22 that is an X-ray emission device. Thus, the X-ray generation apparatus 100 may be configured to include the electron emission device 10 including the plurality of electron emission sources 16 and the X-ray emission unit 20 including the anode electrode 22 that is the X-ray emission device.

The X-ray detection apparatus 200 includes a plurality of X-ray detection units 210 that may be independently driven. The X-ray detection units 210 may correspond to the X-ray emission units 21 respectively.

The X-ray emission units 21 and the X-ray detection units 210 may correspond to each other one-to-one or. Each of the X-ray emission units 21 may correspond to two or more of the X-ray detection units 21 or each of two or more of the X-ray emission units 21 may also correspond to each of the X-ray detection units 210.

The X-ray imaging system according to the above-described embodiment includes a flat panel type X-ray generation apparatus. Thus, an object is disposed between the flat panel type X-ray generation apparatus and an X-ray detection apparatus, thereby implementing the X-ray imaging system having a very small thickness. A limited part of the X-ray generation apparatus may be partially driven to generate an X-ray, thereby photographing a specific region of the object, and such selectively partial photographing prevents an irradiation of the X-ray to an unnecessary region, thereby reducing an exposure rate. The X-ray generation apparatus may include an electron emission device including a plurality of electron emission units that are independently driven. The X-ray generation apparatus may include a plurality of X-ray emission units that are independently driven and generate the X-ray by electrons emitted by the electron emission device. At least a part of the plurality of electron emission units may be sequentially driven, thereby 3-dimensionally photographing the specific region of the object. A cooling apparatus may be used to reduce heating in an anode electrode to which a high voltage is applied, and a high voltage stability may increase in proximity photographing of the object by using an insulating layer. A transmission loss may be reduced through an X-ray transmission window provided in a vacuum chamber.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

1. An X-ray generation apparatus comprising:
an electron emission device comprising a plurality of electron emission units that are independently driven and emit electrons;
a transmission type X-ray emission unit for emitting an X-ray by electrons emitted by the plurality of electron emission units; and
a vacuum chamber for shielding the electron emission device and the transmission type X-ray emission unit by using vacuum.

2. The X-ray generation apparatus of claim 1, wherein an X-ray transmission window that radiates the X-ray emitted by the X-ray emission unit to the outside of the vacuum chamber is provided in the vacuum chamber.

3. The X-ray generation apparatus of claim 2, wherein the X-ray transmission window comprises Be, C, Al, or a metal alloy including at least one of Be, C, and Al.

4. The X-ray generation apparatus of claim 1, wherein one or more of the plurality of electron emission units are simultaneously or sequentially driven to emit the electrons, and
wherein the X-ray emission unit comprises a plurality of X-ray emitters that emit the X-ray by the electrons emitted by the plurality of electron emission units.

5. The X-ray generation apparatus of claim 1, wherein the X-ray emission unit comprises an anode electrode that generates the X-ray by the electrons emitted by the plurality of electron emission units.

6. The X-ray generation apparatus of claim 5, wherein the anode electrode comprises an anode substrate and a coating layer provided on one surface of the anode substrate, and
wherein the coating layer comprises W, Mo. Ag, Cr, Fe, Co, Cu or a metal alloy including at least one of W, Mo. Ag, Cr, Fe, Co, and Cu.

7. The X-ray generation apparatus of claim 6, wherein the anode substrate is a carbon substrate, and
wherein the coating layer is a tungsten coating layer.

8. The X-ray generation apparatus of claim 5, further comprising: an insulating layer for separating the X-ray emission unit from the vacuum chamber.

9. The X-ray generation apparatus of claim 5, further comprising: a cooling apparatus spaced apart from the vacuum chamber by an insulating layer and cooling heat generated by the anode electrode.

10. The X-ray generation apparatus of claim 7, wherein the insulating layer comprises ceramics or plastics.

11. The X-ray generation apparatus of claim 1, wherein the vacuum chamber comprises a getter pump that removes a gas from the inside thereof.

12. The X-ray generation apparatus of claim 1, wherein each of the plurality of electron emission units comprises:
a gate insulating layer provided on a substrate and forming a cavity connected to the outside by an opening through which the electrons are emitted;
a cathode electrode disposed in the cavity;
an electron emission source disposed on the cathode electrode; and
a gate electrode on the gate insulating layer.

13. The X-ray generation apparatus of claim 12, wherein each of the plurality of electron emission units comprises a carbon nanotube (CNT), a carbon nanofiber, metal, silicon, an oxide, diamond, diamond like carbon (DLC), a carbide compound or a nitrogen compound.

14. The X-ray generation apparatus of claim 12, wherein one or more of the plurality of electron emission units are simultaneously or sequentially driven to emit the electrons.

15. An X-ray imaging system comprising:
an X-ray generation apparatus of claim 1; and
an X-ray detection apparatus for detecting an X-ray that is irradiated from the X-ray generation apparatus and passes through an object.

16. The X-ray imaging system of claim 15, wherein the X-ray detection apparatus comprises a plurality of X-ray detection units that are 2-dimensionally arranged and independently driven.

17. The X-ray imaging system of claim 15, wherein an X-ray transmission window that radiates the X-ray emitted by the X-ray emission unit to the outside of the vacuum chamber is provided in the vacuum chamber, and the X-ray transmission window comprises Be, C, Al, or a metal alloy including at least one of Be, C, and Al.

18. The X-ray imaging system of claim 17, wherein the X-ray emission unit comprises an anode electrode that generates the X-ray by the electrons emitted by the plurality of electron emission units,
wherein the anode electrode comprises an anode substrate and a coating layer provided on one surface of the anode substrate, and
wherein the coating layer comprises W, Mo. Ag, Cr, Fe, Co, Cu or a metal alloy including at least one of W, Mo. Ag, Cr, Fe, Co, and Cu.

19. The X-ray imaging system of claim 18, further comprising: an insulating layer for separating the X-ray emission unit from the vacuum chamber.

20. The X-ray imaging system of claim 19, further comprising: a cooling apparatus spaced apart from the vacuum chamber by an insulating layer and cooling heat generated by the anode electrode.
